(19) United States
(12) Patent Application Publication
(10) Pub. No.: US 2025/0084080 A1
(43) Pub. Date: Mar. 13, 2025

(54) CRYSTALLINE AND SALT FORMS OF AN NLRP3 INHIBITOR

(71) Applicant: BioAge Labs, Inc., Richmond, CA (US)

(72) Inventor: Sanjeev Hukmichand Kothari, Richmond, CA (US)

(21) Appl. No.: 18/882,356

(22) Filed: Sep. 11, 2024

Related U.S. Application Data
(60) Provisional application No. 63/582,161, filed on Sep. 12, 2023.

(51) Int. Cl. C07D 471/04; A61K 31/437

(52) U.S. Cl. CPC C07D 471/04; A61K 31/437

(57) ABSTRACT

This disclosure provides crystalline forms of an NLRP3 inhibitor, and methods of making and using these forms.

### RELATED APPLICATIONS

This application claims priority to U.S. Provisional Application No. 63/582,161 filed Sep. 12, 2023, the entire content of which is hereby incorporated in its entirety.

### BACKGROUND

Aging frailty poses a very concerning problem for the overall health and well-being of individuals and is characterized as a syndrome of multisystem physiological dysregulation. Aging frailty is a geriatric syndrome characterized by weakness, low physical activity, slowed motor performance, exhaustion, and unintentional weight loss (Yao, X. et al., Clinics in Geriatric Medicine 27(1): 79-87 (2011)). Furthermore, there are many studies showing a direct correlation between aging frailty and inflammation (Hubbard, R. E., et al., Biogerontology 11(5):635-641 (2010)). Immunosenescence is characterized by a low grade, chronic systemic inflammatory state known as inflammaging (Franceshi, C. et al., Annals of the New York Academy of Sciences 908:244-254 (2000)). This heightened inflammatory state or chronic inflammation found in aging and aging frailty leads to immune dysregulation and a complex remodeling of both innate and adaptive immunity.

Inhibiting the NLRP3 inflammasome, an oligomeric protein complex that includes ASC and caspase-1, mediates inflammation in an extensive number of preclinical models (Schwaid, A. G., J. Med. Chem. 2021, 64(1), 101-122). At the same time, the NLRP3 inflammasome is part of a larger pro-inflammatory pathway, whose modulation is also being explored. NLRP3 is an inflammasome sensor protein that has been well studied in a number of disease contexts. Many different indications are associated with the NLRP3 inflammasome including diseases related to aging, cryopyrin-associated periodic syndrome (CAPS), nonalcoholic steatohepatitis (NASH), gout, coronary artery disease, Crohn's disease, osteoarthritis, rheumatoid arthritis, Alzheimer's disease, Parkinson's disease, intestinal disorders, acute respiratory distress syndrome (ARDS), amyotrophic lateral sclerosis (ALS), cancer, and dermatological diseases.

Inflammation, as well as activation of the NLRP3 inflammasome, has also been shown to result in hearing loss (Nakanishi, H., et al., Frontiers in Neurology, 2020, 11, 1-7; Nakanishi, H., et al., PNAS, 2017, E7766-E7775). The inflammation-related hearing loss can be age-dependent (Fischer, N., et al., Gerontology, 2019, 1-7), noise-induced (Le Prell, C. G., et al. Current Opinion in Physiology, 2020, 18, 32-36), and the result of a viral infection such as Zika virus and coronavirus (Yee, K. T., et al., Hearing Research, 2020, 395, 1-15).

The NLRP3 inflammasome is therefore a promising drug target. The breadth of the indications it is implicated in speak to the need for therapeutics that target the NLRP3 inflammasome.

### SUMMARY

Provided herein are crystalline forms that inhibit the NLRP3 inflammasome. As such, these compounds are useful in the treatment of a variety of indications, including inflammaging and inflammation.

In a particular aspect, provided herein are crystalline forms of 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide referred to herein as Compound 1:

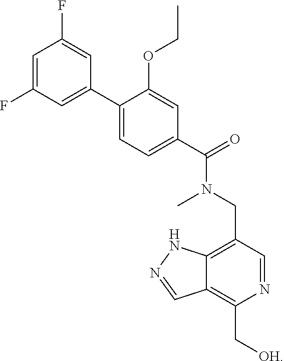 [SMILES: CCOc1cc(C(=O)N(C)Cc2cnc(CO)c3cn[nH]c23)ccc1-c1cc(F)cc(F)c1]

In another aspect, provided herein is a method of treating diseases and disorders associated with the NLRP3 inflammasome in a subject in need thereof comprising administering to the subject a crystalline form 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide.

### BRIEF DESCRIPTION OF THE DRAWINGS

FIG. 1 shows the XRPD diffractogram of crystalline Compound 1 (Pattern B).

FIG. 2 shows the DSC thermogram of crystalline Compound 1 (Pattern B).

FIG. 3 shows the XRPD diffractogram of crystalline Compound 1 monohydrate (Pattern A).

FIG. 4 shows the DSC thermogram of crystalline Compound 1 monohydrate (Pattern A).

FIG. 5 shows the TGA thermogram of crystalline Compound 1 monohydrate (Pattern A).

FIG. 6 shows the XRPD diffractogram of crystalline Compound 1 HCl salt (Pattern A).

FIG. 7 shows the DSC thermogram of crystalline Compound 1 HCl salt (Pattern A).

FIG. 8 shows the XRPD diffractogram of crystalline Compound 1 phosphoric acid salt (Pattern A).

FIG. 9 shows the DSC thermogram of crystalline Compound 1 phosphoric acid salt (Pattern A).

FIG. 10 shows the TGA thermogram of crystalline Compound 1 phosphoric acid salt (Pattern A).

FIG. 11 shows the XRPD diffractogram of crystalline Compound 1 sodium salt (Pattern A).

FIG. 12 shows the DSC thermogram of crystalline Compound 1 sodium salt (Pattern A).

FIG. 13 shows the TGA thermogram of crystalline Compound 1 sodium salt (Pattern A).

### DETAILED DESCRIPTION

The solid state of a compound can be important when the compound is used for pharmaceutical purposes. The physical properties of a compound can change from one solid form to another, which can affect the suitability of the form for pharmaceutical use. For example, a particular crystalline solid compound can overcome the disadvantage of other solid forms of the compound such as, e.g., instability and/or reduced purity.

Provided herein are solid, crystalline forms of 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide (Compound 1):

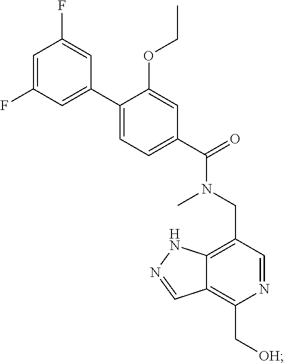 [SMILES: CCOc1cc(C(=O)N(C)Cc2cnc(CO)c3cn[nH]c23)ccc1-c1cc(F)cc(F)c1]

or a pharmaceutically acceptable salt or hydrate thereof.

This compound is disclosed in International Application No. PCT/US2022/021461, the entire content of which is incorporated herein by reference.

The crystalline forms provided herein can be characterized by X-ray powder diffraction (XRPD), differential scanning calorimetry (DSC), and thermogravimetric analysis (TGA).

### Definitions

Listed below are definitions of various terms used to describe the crystalline forms provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used to herein, the term “EC₅₀” refers to the concentration of a compound required to achieve an effect that is 50% of the maximal observed effect of a compound.

As used herein, the term “pharmaceutically acceptable carrier” refers to a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient.

Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound provided herein and are physiologically acceptable to the patient. Supplementary active compounds may also be incorporated into the compositions. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

As used herein, the phrases “therapeutically effective dose” and “therapeutically effective amount” refer to an amount of a compound that prevents the onset, alleviates the symptoms, stops the progression of a disease, or results in another desired biological outcome such as, e.g., improved clinical signs.

The term “treat,” “treated,” “treating,” or “treatment” includes the diminishment or alleviation of at least one symptom associated or caused by the state, disorder or disease being treated.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. In an embodiment, the patient, subject, or individual is human.

The term “administering” or “administration” and the like, refers to providing a therapeutic agent, such as a crystalline form disclosed herein, to the subject in need of treatment. In an embodiment, the subject is a mammal. In another embodiment, the subject is a human.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

### Characterization of Crystalline Forms

In certain embodiments, the crystalline forms described herein are identifiable on the basis of characteristic peaks in an X-ray powder diffraction analysis. X-ray powder diffraction (XRPD) is a scientific technique using X-ray, neutron, or electron diffraction on powder, microcrystalline, or other solid materials for structural characterization of solid materials. A description of the methods used to obtain certain XRPD diffractograms in connection with the crystalline forms provided herein can be found in the Examples below. In an embodiment, the X-ray powder diffraction data provided herein is obtained by a method utilizing Cu Kα radiation.

In an aspect, provided herein is a crystalline form of 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide, or a pharmaceutically acceptable salt or hydrate thereof.

In an embodiment, the crystalline form is a monohydrate. In another embodiment, the crystalline form is an anhydrate.

In yet another embodiment, the crystalline form is a pharmaceutically acceptable salt. In still another embodiment, the pharmaceutically acceptable salt is selected from the group consisting of hydrochloric acid salt, phosphoric acid salt, and sodium salt.

### Free Form Anhydrous

In an embodiment, the crystalline form of Compound 1 is anhydrous.

In an embodiment, the crystalline form is characterized by an XRPD diffractogram having peaks expressed in degrees-2-theta at angles (±0.2 degrees) of 7.2, 20.4, and 20.6.

In another embodiment, the crystalline form is characterized by an XRPD diffractogram having peaks expressed in degrees-2-theta at angles (±0.2 degrees) of 7.2, 20.4, 20.6, and 21.6. In yet another embodiment, the crystalline form is characterized by an XRPD diffractogram having peaks expressed in degrees-2-theta at angles (±0.2 degrees) of 7.2, 11.1, 11.8, 20.4, 20.6, and 21.6. In yet another embodiment, the crystalline form is characterized by an XRPD diffractogram having peaks expressed in degrees-2-theta at angles (±0.2 degrees) of 5.5, 7.2, 11.1, 11.8, 15.3, 20.4, 20.6, 21.6, 26.7, and 30.9.

In an embodiment, the crystalline form is characterized by an XRPD diffractogram having peaks selected from Table 1 (Pattern B).

TABLE 1
XRPD peaks table of free form Pattern B

| Diffraction angle | |
|---|---|
| (°, 2 theta) | Rel. Intensity |
| 5.09 | 10.80% |
| 5.546 | 14.30% |
| 7.193 | 48.80% |
| 10.19 | 4.30% |
| 11.079 | 29.10% |
| 11.786 | 23.10% |
| 12.589 | 8.90% |
| 14.394 | 2.30% |
| 15.288 | 19.40% |
| 15.801 | 3.00% |
| 16.667 | 7.20% |
| 18.083 | 2.20% |
| 20.432 | 100.00% |
| 20.573 | 45.50% |
| 21.64 | 41.70% |
| 22.236 | 2.80% |
| 22.733 | 2.00% |
| 23.554 | 1.80% |
| 24.158 | 2.70% |
| 25.369 | 2.10% |
| 25.595 | 3.30% |
| 26.698 | 15.10% |
| 27.033 | 12.40% |
| 28.805 | 0.70% |
| 30.87 | 15.30% |
| 31.746 | 1.80% |
| 36.079 | 4.50% |
| 36.98 | 1.60% |

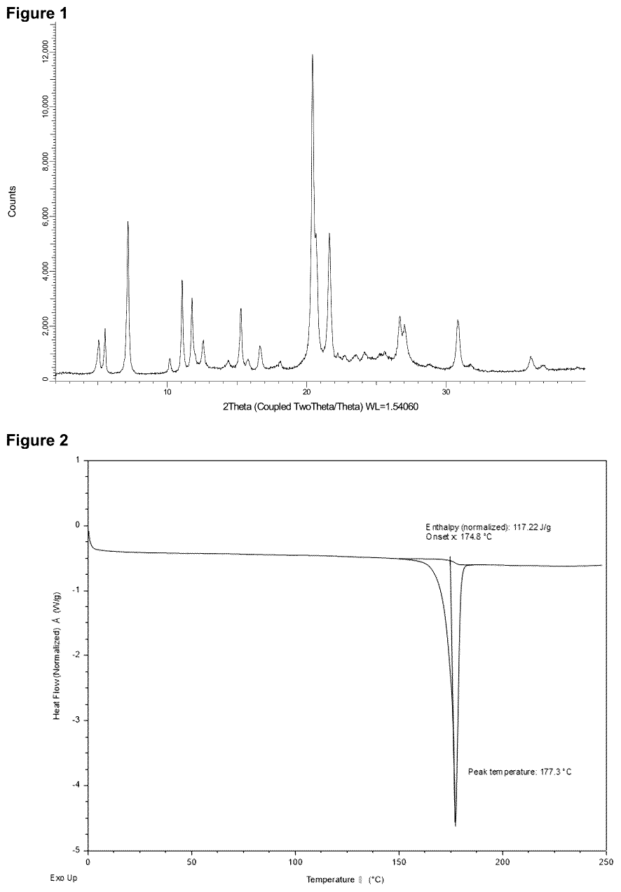

In another embodiment, the crystalline form has the XRPD diffractogram substantially as depicted in FIG. 1.

In still another embodiment, the crystalline form is a monohydrate having a DSC thermogram characterized by an endotherm with an onset temperature of 177.3° C. In another embodiment, the crystalline form has a DSC thermogram substantially as depicted in FIG. 2.

### Free Form Monohydrate

In an embodiment, the crystalline form is a monohydrate characterized by an XRPD diffractogram having peaks expressed in degrees-2-theta at angles (±0.2 degrees) of 6.5, 12.9, and 16.1. In another embodiment, the crystalline form is characterized by an XRPD diffractogram having peaks expressed in degrees-2-theta at angles (±0.2 degrees) of 6.5, 11.2, 12.9, and 16.1. In yet another embodiment, the crystalline form is characterized by an XRPD diffractogram having peaks expressed in degrees-2-theta at angles (±0.2 degrees) of 6.5, 11.2, 12.9, 16.1, 22.7, 24.8, and 32.6.

In an embodiment, the crystalline form is characterized by an XRPD diffractogram having peaks selected from Table 2 (Pattern A).

TABLE 2
XRPD peaks table of free form Pattern A

| Diffraction angle | |
|---|---|
| (°2 theta) | Rel. Intensity |
| 3.25 | 3.10% |
| 5.832 | 0.10% |
| 6.477 | 98.20% |
| 8.801 | 1.40% |
| 9.196 | 0.20% |
| 9.57 | 2.10% |
| 9.697 | 3.70% |
| 10.6 | 0.20% |
| 11.222 | 34.50% |
| 11.977 | 0.60% |
| 12.869 | 55.50% |
| 12.924 | 100.00% |
| 13.471 | 3.00% |
| 16.156 | 40.30% |
| 17.613 | 4.30% |
| 17.936 | 2.70% |
| 18.389 | 1.80% |
| 18.875 | 1.30% |
| 19.406 | 0.20% |
| 19.632 | 0.20% |
| 20.645 | 0.30% |
| 21.238 | 5.60% |
| 21.818 | 0.20% |
| 22.675 | 19.40% |
| 23.224 | 3.30% |
| 23.838 | 0.20% |
| 24.842 | 24.30% |
| 25.509 | 0.40% |
| 25.944 | 0.20% |
| 26.511 | 0.30% |
| 26.944 | 0.70% |
| 27.066 | 0.70% |
| 27.707 | 2.40% |
| 27.951 | 4.60% |
| 28.857 | 0.40% |
| 29.264 | 5.00% |
| 29.668 | 0.40% |
| 30.097 | 0.20% |
| 30.307 | 0.70% |
| 32.087 | 0.70% |
| 32.586 | 19.50% |
| 33.246 | 0.60% |
| 34.037 | 0.30% |
| 34.35 | 0.20% |
| 35.943 | 3.20% |
| 36.543 | 0.80% |
| 37.552 | 0.20% |
| 38.608 | 0.70% |
| 39.274 | 0.30% |
| 39.809 | 0.70% |

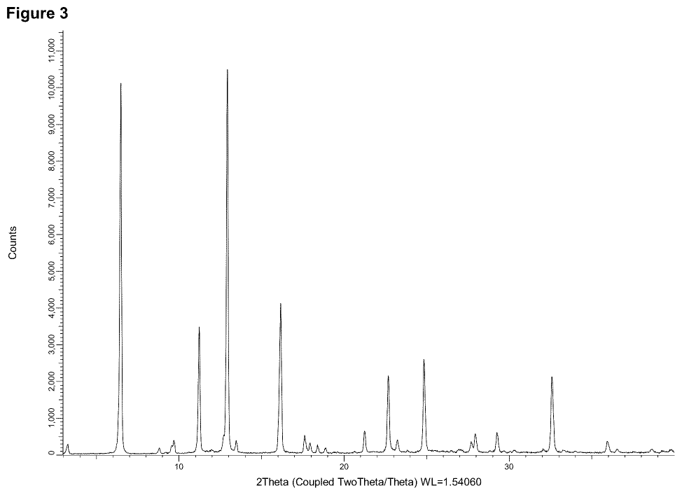

In another embodiment, the crystalline form has the XRPD diffractogram substantially as depicted in FIG. 3.

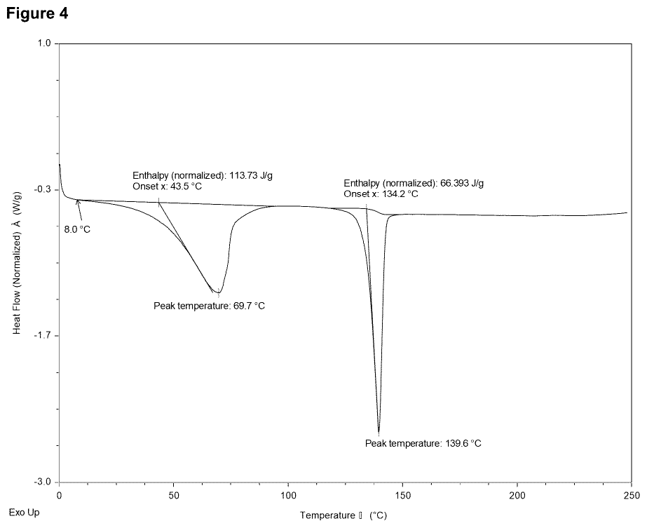

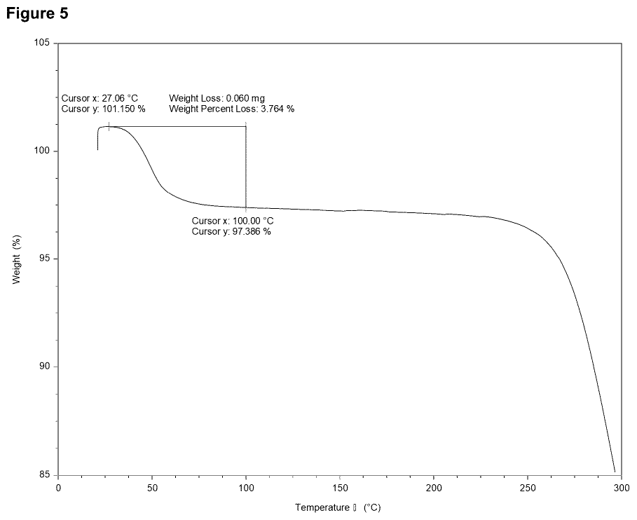

In still another embodiment, the crystalline form is a monohydrate having a DSC thermogram characterized by an endotherm with an onset temperature of 139.6° C. In another embodiment, the crystalline form has a DSC thermogram substantially as depicted in FIG. 4. In yet another embodiment, the crystalline form has a TGA thermogram substantially as depicted in FIG. 5.

### Hydrochloric Acid Salt

In an embodiment, the crystalline form is 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide hydrochloric acid salt. In another embodiment, the crystalline form of the HCl salt is characterized by an XRPD diffractogram having peaks expressed in degrees-2-theta at angles (±0.2 degrees) of 12.3, 18.1, and 18.2. In yet another embodiment, the crystalline form of the HCl salt is characterized by an XRPD diffractogram having peaks expressed in degrees-2-theta at angles (±0.2 degrees) of 12.3, 12.8, 18.1, 18.2, and 28.7. In still another embodiment, the crystalline form of the HCl salt is characterized by an XRPD diffractogram having peaks expressed in degrees-2-theta at angles (±0.2 degrees) of 12.3, 12.8, 18.1, 18.2, 26.0, and 28.7.

In an embodiment, the crystalline form is characterized by an XRPD diffractogram having peaks selected from Table 3 (HCl salt of Pattern A).

TABLE 3
XRPD peaks table of HCl salt Pattern A

| Diffraction angle (°, 2 theta) | Rel. Intensity |
|---|---|
| 3.221 | 2.20% |
| 6.443 | 7.60% |
| 9.157 | 1.90% |
| 9.602 | 1.50% |
| 10.117 | 0.80% |
| 10.392 | 1.60% |
| 12.29 | 100.00% |
| 12.817 | 15.00% |
| 14.158 | 1.10% |
| 14.628 | 0.70% |
| 16.023 | 2.00% |
| 16.739 | 0.90% |
| 17.283 | 9.30% |
| 18.12 | 16.30% |
| 18.27 | 16.70% |
| 19.23 | 0.30% |
| 20.187 | 6.10% |
| 21.829 | 6.90% |
| 22.467 | 5.80% |
| 23.025 | 2.20% |
| 23.224 | 2.40% |
| 23.177 | 2.10% |
| 23.794 | 5.10% |
| 25.39 | 5.50% |
| 26.042 | 10.60% |
| 27.312 | 3.20% |
| 28.045 | 2.30% |
| 28.723 | 13.50% |
| 29.114 | 5.80% |
| 29.466 | 3.80% |
| 29.873 | 2.40% |
| 30.47 | 1.80% |
| 31.406 | 1.40% |
| 31.623 | 2.00% |
| 32.258 | 6.10% |
| 33.246 | 0.90% |
| 33.915 | 1.40% |
| 34.835 | 1.20% |
| 35.12 | 0.50% |
| 35.527 | 0.20% |
| 36.601 | 1.50% |
| 38.219 | 2.80% |
| 39.347 | 1.90% |
| 39.759 | 0.60% |

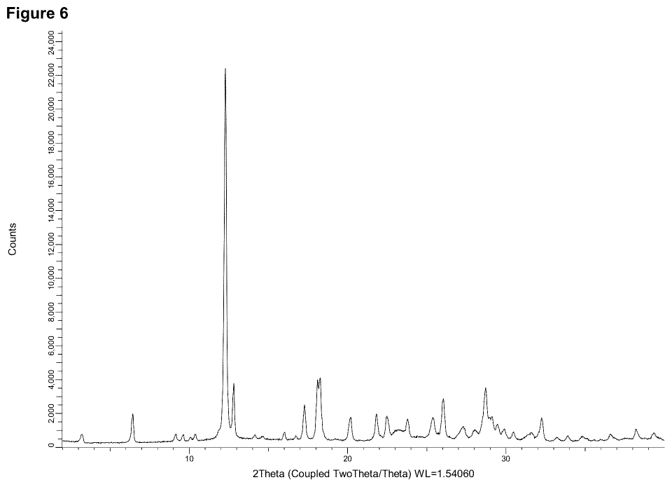

In another embodiment, the crystalline form has the XRPD diffractogram substantially as depicted in FIG. 6.

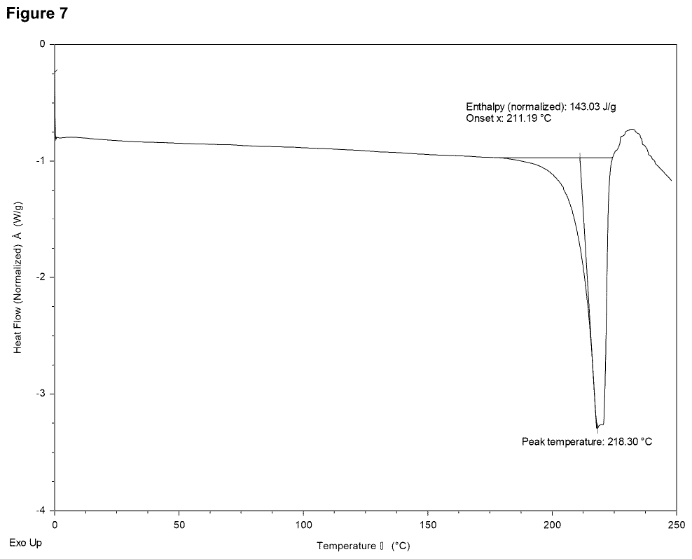

In still another embodiment, the crystalline form is an HCl salt having a DSC thermogram characterized by an endotherm with an onset temperature of 218.3° C. In another embodiment, the crystalline form has a DSC thermogram substantially as depicted in FIG. 7.

### Phosphoric Acid Salt

In an embodiment, the crystalline form is 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide phosphoric acid salt.

In another embodiment, the crystalline form of the phosphoric acid salt is characterized by an XRPD diffractogram having peaks expressed in degrees-2-theta at angles (±0.2 degrees) of 3.1, 9.2, and 16.4. In yet another embodiment, the crystalline form of the phosphoric acid salt is characterized by an XRPD diffractogram having peaks expressed in degrees-2-theta at angles (±0.2 degrees) of 3.1, 9.2, 11.7, 13.9, 15.4, 16.4, and 24.7. In still another embodiment, the crystalline form of the phosphoric acid salt is characterized by an XRPD diffractogram having peaks expressed in degrees-2-theta at angles (±0.2 degrees) of 3.1, 9.2, 11.7, 13.9, 15.4, 16.4, 18.1, 19.0, 16.4, and 24.7.

In an embodiment, the crystalline form is characterized by an XRPD diffractogram having peaks selected from Table 4 (phosphoric acid salt of Pattern A).

TABLE 4
XRPD peaks table of phosphate salt Pattern A

| Diffraction angle (°, 2 theta) | Rel. Intensity |
|---|---|
| 3.103 | 100.00% |
| 9.227 | 60.30% |
| 10.064 | 7.30% |
| 11.701 | 35.80% |
| 12.289 | 6.70% |
| 13.898 | 34.50% |
| 15.366 | 41.70% |
| 16.376 | 60.00% |
| 17.82 | 5.90% |
| 18.101 | 16.40% |
| 19.048 | 16.10% |
| 20.145 | 11.90% |
| 21.056 | 1.70% |
| 21.564 | 7.20% |
| 21.809 | 10.00% |
| 22.494 | 4.10% |
| 23.259 | 1.30% |
| 23.693 | 13.70% |
| 24.715 | 43.60% |
| 25.847 | 4.00% |
| 26.454 | 1.30% |
| 26.882 | 1.30% |
| 27.211 | 3.20% |
| 27.834 | 19.20% |
| 28.546 | 2.80% |
| 30.464 | 4.90% |
| 30.725 | 3.30% |
| 31.243 | 1.00% |
| 32.026 | 1.00% |
| 32.357 | 0.70% |
| 33.888 | 1.90% |
| 34.126 | 5.90% |
| 36.995 | 0.90% |
| 37.11 | 0.90% |
| 37.315 | 1.60% |
| 38.03 | 2.10% |
| 38.822 | 0.60% |

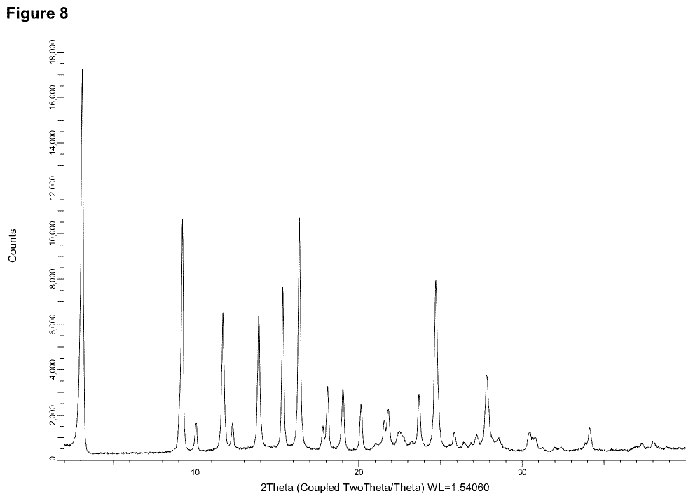

In another embodiment, the crystalline form has the XRPD diffractogram substantially as depicted in FIG. 8.

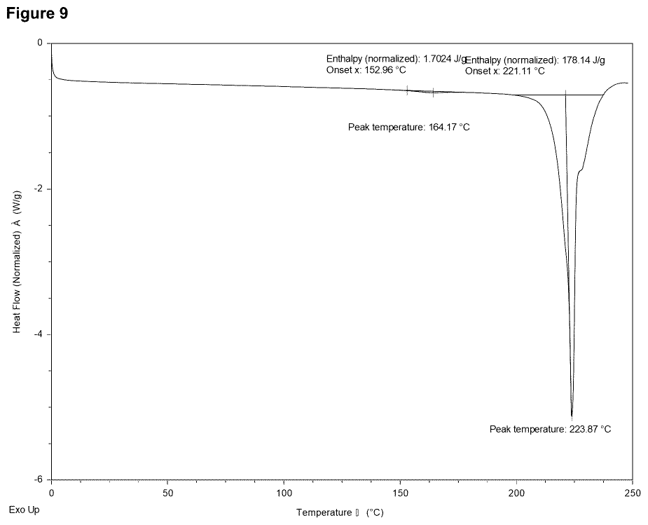

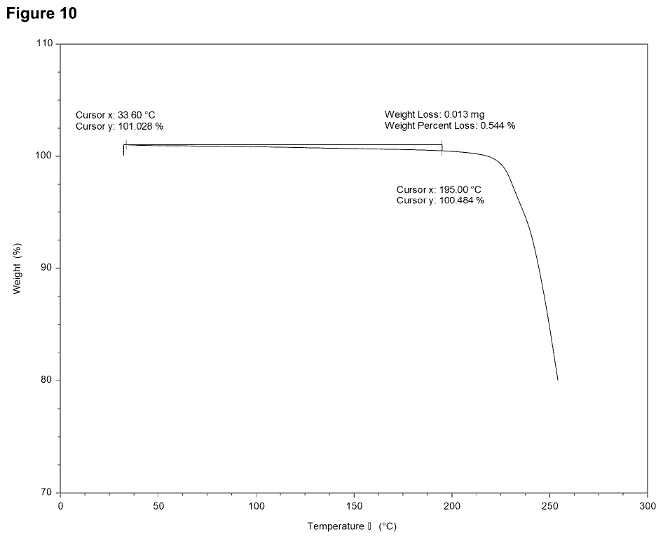

In another embodiment, the crystalline form is a phosphoric acid salt having a DSC thermogram characterized by an endotherm with an onset temperature of 223.9° C. In another embodiment, the crystalline form has a DSC thermogram substantially as depicted in FIG. 9. In yet another embodiment, the crystalline form has a TGA thermogram substantially as depicted in FIG. 10.

### Sodium Salt

In an embodiment, the crystalline form is 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide sodium salt.

In another embodiment, the crystalline form of the sodium salt is characterized by an XRPD diffractogram having peaks expressed in degrees-2-theta at angles (±0.2 degrees) of 10.3, 21.1, and 25.8. In yet another embodiment, the crystalline form of the sodium salt is characterized by an XRPD diffractogram having peaks expressed in degrees-2-theta at angles (±0.2 degrees) of 10.3, 17.3, 20.6, 21.1, 23.3, and 25.8. In still another embodiment, the crystalline form of the sodium salt is characterized by an XRPD diffractogram having peaks expressed in degrees-2-theta at angles (±0.2 degrees) of 10.3, 14.9, 17.2, 17.3, 18.8, 20.6, 20.0, 21.1, 23.3, and 25.8.

In an embodiment, the crystalline form is characterized by an XRPD diffractogram having peaks selected from Table 5 (sodium salt of Pattern A).

TABLE 5
XRPD peaks table of sodium salt Pattern A

| Diffraction angle (°, 2 theta) | Rel. Intensity |
|---|---|
| 5.164 | 83.30% |
| 10.282 | 67.80% |
| 11.318 | 1.00% |
| 11.953 | 24.90% |
| 13.444 | 17.50% |
| 14.903 | 33.10% |
| 15.417 | 12.90% |
| 15.738 | 3.50% |
| 16.946 | 13.20% |
| 17.155 | 28.90% |
| 17.342 | 36.20% |
| 18.83 | 27.50% |
| 20.014 | 29.10% |
| 20.578 | 58.30% |
| 21.124 | 77.10% |
| 21.608 | 4.00% |
| 22.222 | 18.00% |
| 22.669 | 3.20% |
| 23.267 | 38.30% |
| 23.69 | 13.50% |
| 23.955 | 10.40% |
| 24.299 | 1.80% |
| 24.57 | 5.40% |
| 25.019 | 5.90% |
| 25.365 | 3.30% |
| 25.775 | 100.00% |
| 26.598 | 12.10% |
| 26.836 | 3.00% |
| 27.036 | 1.80% |
| 27.409 | 3.60% |
| 27.665 | 2.30% |
| 27.989 | 18.70% |
| 29.039 | 4.00% |
| 29.238 | 1.70% |
| 29.99 | 8.00% |
| 30.32 | 10.40% |
| 31.038 | 12.10% |
| 31.684 | 1.20% |
| 32.116 | 1.20% |
| 32.4 | 10.50% |
| 32.932 | 7.00% |
| 33.452 | 2.40% |
| 33.688 | 2.70% |
| 34.257 | 7.80% |
| 34.603 | 1.00% |
| 35.55 | 1.70% |
| 35.817 | 1.40% |
| 36.226 | 2.90% |
| 36.345 | 3.60% |
| 36.807 | 1.20% |
| 37.159 | 2.60% |
| 37.243 | 2.50% |
| 37.628 | 1.40% |
| 38.079 | 2.50% |
| 39.314 | 1.30% |
| 39.597 | 1.60% |

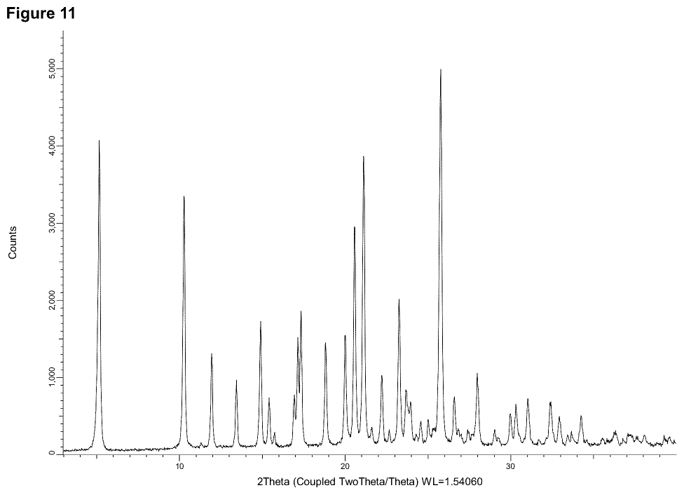

In another embodiment, the crystalline form has the XRPD diffractogram substantially as depicted in FIG. 11.

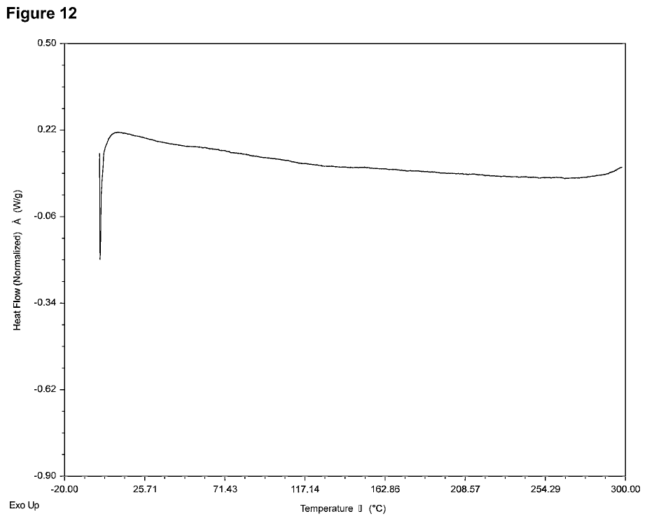

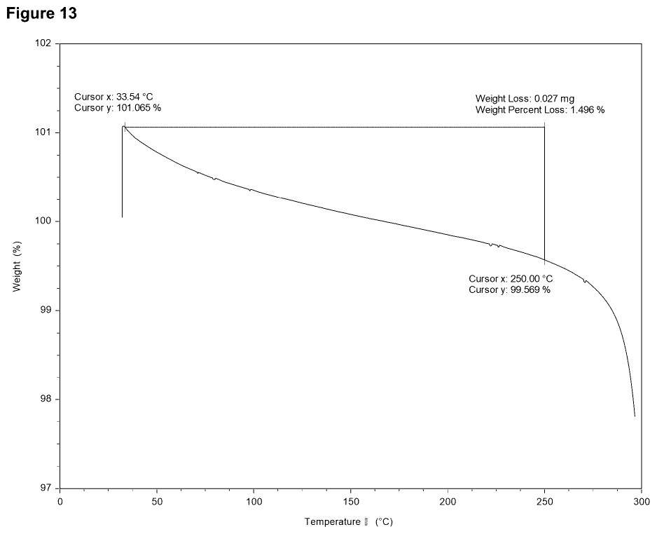

In another embodiment, the crystalline form has a DSC thermogram substantially as depicted in FIG. 12. In yet another embodiment, the crystalline form has a TGA thermogram substantially as depicted in FIG. 13.

### Pharmaceutically Acceptable Salts

In another aspect, provided herein is a crystalline form of a pharmaceutically acceptable salt of 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide, wherein the pharmaceutically acceptable salt is selected from the group consisting of hydrochloric acid salt, phosphoric acid salt, and sodium salt.

In an embodiment, the pharmaceutically acceptable salt is hydrochloric acid salt. In another embodiment, the pharmaceutically acceptable salt is phosphoric acid salt. In yet another embodiment, the pharmaceutically acceptable salt is sodium salt.

In an embodiment, the X-ray powder diffraction (XRPD) of the crystalline forms herein are measured by Cu Kα radiation operating at 40 kV, 40 mA. In another embodiment of the XRPD, scans are run from 2-40 degrees 2-theta with a step size of 0.02 degrees and a scan time of 0.3 second per step.

In yet another aspect, provided herein is a pharmaceutical composition comprising a crystalline form of the present disclosure and a pharmaceutically acceptable carrier.

The pharmaceutical preparations disclosed herein can be prepared in accordance with standard procedures and are administered at dosages that are selected to reduce, prevent or eliminate disease. See, for example, Remington's Pharmaceutical Sciences, Mack Publishing Company, Easton, PA and Goodman and Gilman's “The Pharmaceutical Basis of Therapeutics,” Pergamon Press, New York, NY, the contents of which are incorporated herein by reference, for a general description of the methods for administering various agents for human therapy.

The pharmaceutical compositions described herein can comprise a crystalline form disclosed herein in association with one or more nontoxic, pharmaceutically acceptable carriers and/or diluents and/or adjuvants and/or excipients.

For oral or parenteral administration, the crystalline form disclosed herein can be mixed with conventional pharmaceutical carriers and excipients and used in the form of tablets, capsules, elixirs, suspensions, syrups, wafers and the like. The compositions comprising a crystalline form disclosed herein can contain from about 0.1% to about 99% by weight of the active compound, such as from about 10% to about 30%.

For oral use, solid formulations such as tablets and capsules are useful. Sustained release or enterically coated preparations can also be devised. For pediatric and geriatric applications, one embodiment provides suspensions, syrups and chewable tablets. For oral administration, the pharmaceutical compositions are in the form of, for example, a tablet, capsule, suspension or liquid.

The pharmaceutical compositions can be made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, fillers, lubricants, disintegrants, or acceptable wetting agents. Oral liquid preparations generally are in the form of aqueous or oily solutions, suspensions, emulsions, syrups or elixirs.

The pharmaceutical compositions disclosed herein can be placed in a pharmaceutically acceptable carrier and are delivered to a recipient subject (e.g., a human) in accordance with known methods of drug delivery. In general, the methods of delivering the pharmaceutical compositions in vivo utilize art-recognized protocols for delivering the agent with the only substantial procedural modification being the substitution of a crystalline form of the present disclosure for the drugs in the art-recognized protocols.

### Methods of Treatment

Provided herein are methods for the treatment of a disease comprising administering a crystalline form of 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide, or a pharmaceutical composition comprising the crystalline form and a pharmaceutically acceptable carrier.

In an aspect, provided is a method of inhibiting NLRP3 inflammasome in a subject in need thereof comprising administering to the subject a therapeutically effective amount of a crystalline form disclosed herein.

In another aspect, provided is a method of treating inflammation in a subject in need thereof comprising administering to the subject a therapeutically effective amount of a crystalline form disclosed herein.

In yet another aspect, provided is a method of treating inflammaging in a subject in need thereof comprising administering to the subject a therapeutically effective amount of a crystalline form disclosed herein.

In still another aspect, provided is a method of treating cryopyrin-associated periodic syndrome (CAPS) in a subject in need thereof comprising administering to the subject a therapeutically effective amount of a crystalline form disclosed herein.

In an embodiment, the CAPS is selected from the group consisting of familial cold autoinflammatory syndrome, Muckle-Wells syndrome, and neonatal-onset multisystem inflammatory disease.

In an aspect, provided is a method of treating a disease or disorder of the inner ear in a subject in need thereof comprising administering to the subject a therapeutically effective amount of the crystalline form disclosed herein.

In an embodiment, the disease or disorder of the inner ear is selected from the group consisting of hearing loss, hearing impairment, vertigo, Meniere's disease, and tinnitus.

In another aspect, provided herein is a method of treating a dermatologic disease in a subject in need thereof, comprising administering to the individual a therapeutically effective amount of a crystalline form disclosed herein.

In an embodiment, the dermatologic disease is selected from the group consisting of psoriasis, urticaria, skin photoaging, and eczema.

Also provided herein is a method of using the crystalline forms provided herein for treatment or amelioration of aging or an aging-related condition negatively impacting longevity or quality of life, wherein the aging-related condition negatively impacting longevity or quality of life is selected from the group consisting of inflammation, anemia, hyperglycemia, dyslipidemia, hyperinsulinemia, insulin resistance, immunosuppression, liver disease, iron overload, hypertrigliceridemia, impaired skin integrity, wound healing, scarring, pain, allergies, sleep disorders and problems, gastrointestinal disorders and problems, Th1-type inflammation, Th2-type inflammation, an inflammatory disease involving T-cell dependent B cell proliferation, T-cell dependent B cell proliferation, allergy, asthma, atherosclerosis, autoimmunity, hypercholesterolemia, chronic inflammation, chronic obstructive pulmonary disease (COPD), Crohn's disease, cutaneous responses to tissue damage, fibrosis, hematological oncology, metabolic diseases, cardiovascular disease, organ transplantation, psoriasis, liver fibrosis, dermatitis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and diseases that are driven or exacerbated by one or more factors selected from the group consisting of alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1.alpha., IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNF.alpha.), vascular cell adhesion molecule (VCAM-1), and vascular endothelial growth factor 2 (VEGFR2), comprising: administering to a subject in need thereof a crystalline form provided herein.

In an aspect, provided herein is a method of reversing a normal aging process in subject comprising administering to the subject a therapeutically effective amount of a crystalline form provided herein or a pharmaceutically acceptable salt thereof.

In another aspect, provided herein is a method of reversing a normal aging process in subject comprising administering to the subject a pharmaceutical composition comprising a therapeutically effective amount of a crystalline form provided herein or a pharmaceutically acceptable salt thereof.

In yet another aspect, provided herein is a method of extending lifespan of a subject comprising administering to the subject a therapeutically effective amount of a crystalline form provided herein or a pharmaceutically acceptable salt thereof.

In still another aspect, provided herein is a method of extending lifespan of a subject comprising administering to the subject a pharmaceutical composition comprising a therapeutically effective amount of a crystalline form provided herein or a pharmaceutically acceptable salt thereof.

In another aspect, provided herein is a method to slow down and mitigate the aging process in a subject comprising administering to the subject a therapeutically effective amount of a crystalline form provided herein or a pharmaceutically acceptable salt thereof.

In another aspect, provided herein is a method of inhibiting or modulating the pro-inflammatory pathway in a cell comprising contacting the cell with a crystalline form provided herein, or a pharmaceutically acceptable salt thereof. In yet another aspect, provided herein is a method of inhibiting or modulating NLRP3 in a cell comprising contacting the cell with a crystalline form provided herein, or a pharmaceutically acceptable salt thereof.

Treatment of a cell (in vitro or in vivo) that expresses a NLRP3 inflammasome with a crystalline form provided herein can result in inhibiting the pro-inflammatory pathway and inhibiting downstream events related to the signaling pathway such as inflammation or inflammaging.

In another aspect, provided herein is a method of treating a neurosensory disease in a subject in need thereof, comprising administering to the individual a therapeutically effective amount of a crystalline form disclosed herein.

In an embodiment, the neurosensory disease is selected from the group consisting of hearing loss, hearing injury, and ocular disease. In an embodiment, the ocular disease is retinal and optic nerve injury.

In yet another aspect, provided herein is a method of treating an inflammatory disorder in a subject in need thereof, comprising administering to the subject a therapeutically effective amount of a crystalline form disclosed herein.

In an embodiment, the inflammatory disorder is selected from the group consisting of allergy, asthma, atopic dermatitis, atherosclerosis, autoimmune diseases, coeliac disease, chronic inflammation, glomerulonephritis, hepatitis, inflammatory bowel disease, preperfusion injury, SARS-COV-2 infection, transplant rejection, heart disease, diabetes, arthritis, Crohn's disease, ulcerative colitis, non-alcoholic steatohepatitis (NASH), gout, coronary artery disease, rheumatoid arthritis, intestinal disorders, and acute respiratory distress syndrome (ARDS).

In another embodiment, the inflammatory disorder is a neuroinflammatory disease. In yet another embodiment, the inflammatory disorder is inner ear inflammation.

In an embodiment, a chronic inflammation comprises a tissue inflammation. Tissue inflammation is a chronic inflammation that is confined to a particular tissue or organ. In an embodiment, a tissue inflammation comprises, e.g., a skin inflammation, ocular inflammation, a muscle inflammation, a tendon inflammation, a ligament inflammation, a bone inflammation, a cartilage inflammation, a lung inflammation, a heart inflammation, a liver inflammation, a pancreatic inflammation, a kidney inflammation, a bladder inflammation, a stomach inflammation, an intestinal inflammation, a neuron inflammation, and a brain inflammation.

In another embodiment, a chronic inflammation comprises a systemic inflammation. Although the processes involved are identical to tissue inflammation, systemic inflammation is not confined to a particular tissue but in fact overwhelms the body, involving the endothelium and other organ systems. When it is due to infection, the term sepsis is applied, with the terms bacteremia being applied specifically for bacterial sepsis and viremia specifically to viral sepsis. Vasodilation and organ dysfunction are serious problems associated with widespread infection that may lead to septic shock and death.

In yet another embodiment, a chronic inflammation comprises an arthritis. Arthritis includes a group of conditions involving damage to the joints of the body due to the inflammation of the synovium including, without limitation osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

In still another aspect, provided herein is a method of treating an age-related disorder in a subject in need thereof, comprising administering to the individual a therapeutically effective amount of a crystalline form disclosed herein.

In an embodiment, the age-related disorder is selected from the group consisting of neurodegeneration, cardiovascular disease, insulin resistance, diabetes, osteoporosis, osteoarthritis, cognitive decline, dementia, frailty, cataracts, arthritis, obesity, hypertension, angina, congestive heart failure, dyslipidemia, myocardial infarction, vascular disease, respiratory disease, kidney disease, cerebrovascular disease, peripheral vascular disease, Alzheimer's disease, cardiac diastolic dysfunction, benign prostatic hypertrophy, aortic aneurysm, and emphysema.

In another aspect, provided herein is a method of treating a metabolic condition in a subject in need thereof, comprising administering to the individual a therapeutically effective amount of a crystalline form disclosed herein.

In an embodiment, the metabolic condition is selected from the group consisting of diabetes, obesity, cystic fibrosis, and hyperthyroidism.

In yet another aspect, provided herein is a method of treating a neurodegenerative disease in a subject in need thereof, comprising administering to the individual a therapeutically effective amount of a crystalline form disclosed herein.

In an embodiment, the neurodegenerative disease is selected from the group consisting of Alzheimer's disease, Parkinson's disease, Huntington's disease, multiple sclerosis (MS), amyotrophic lateral sclerosis (ALS), and Batten disease.

In an aspect, provided herein is a method of treating a disease or disorder of the inner ear in a subject in need thereof, comprising administering to the individual a therapeutically effective amount of a crystalline form disclosed herein.

In an embodiment, the disease or disorder of the inner ear is selected from the group consisting of hearing loss, hearing impairment, vertigo, Meniere's disease, and tinnitus. In another embodiment, the disease of the inner ear is hearing loss. In yet another embodiment, the disease of the inner ear is hearing impairment.

In another embodiment, the hearing loss is age-related, noise-induced, or the result of a viral infection. In yet another embodiment, the viral infection is Zika virus or coronavirus.

In another aspect, the disclosure provides a crystalline form disclosed herein, or a pharmaceutically acceptable salt thereof, for use in the manufacture of a medicament for treating or preventing a disease in which NLRP3 inflammasome plays a role.

In an aspect, provided herein is a method of treating a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides crystalline forms of a compound that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer.

Therefore, in an aspect, provided herein is a method of treating cancer in a subject in need thereof comprising administering to the subject a therapeutically effective amount of a crystalline form disclosed herein, or a pharmaceutically acceptable salt thereof.

In an embodiment, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In another embodiment, the cancer is selected from the group consisting of myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

In an embodiment, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In an embodiment, the cancer is selected from the group consisting of tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject crystalline forms include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the crystalline forms described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In another aspect, provided herein is the use of one or more crystalline forms of the disclosure in the manufacture of a medicament for the treatment of cancer, including without limitation the various types of cancer disclosed herein.

In some embodiments, the crystalline forms of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the crystalline forms of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

### Administration/Dosage/Formulations

Actual dosage levels of the active ingredients in the pharmaceutical compositions discussed herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the crystalline form, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed crystalline form at levels lower than that required in order to achieve the desired therapeutic effect and gradually increase the dosage until the desired effect is achieved.

In particular embodiments, it is especially advantageous to formulate the crystalline form in dosage unit form for ease of administration and uniformity of dosage.

“Dosage unit form,” as used herein, refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the crystalline form disclosed herein are dictated by and directly dependent on (a) the unique characteristics of the disclosed compound and the particular therapeutic effect to be achieved, and (b) the limitations inherent in the art of compounding/formulating such a disclosed compound for the treatment of Kennedy's disease (SBMA) in a patient.

In one embodiment, the crystalline form provided herein is formulated using one or more pharmaceutically acceptable excipients or carriers. In one embodiment, the pharmaceutical compositions comprise a therapeutically effective amount of the disclosed crystalline form and a pharmaceutically acceptable carrier.

In some embodiments, the dose of a disclosed compound is from about 1 mg to about 1,000 mg. In some embodiments, a dose of the disclosed compound used in compositions described herein is less than about 1,000 mg, or less than about 800 mg, or less than about 600 mg, or less than about 500 mg, or less than about 300 mg, or less than about 200 mg, or less than about 100 mg, or less than about 50 mg, or less than about 20 mg, or less than about 10 mg. For example, a dose is about 10 mg, 20 mg, 25 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240, 260 mg, 280 mg, 300 mg, 350 mg, 400 mg, 450 mg, 500 mg, 550 mg, or about 600 mg.

Routes of administration of any of the compositions disclosed herein include oral, nasal, rectal, intravaginal, parenteral, buccal, sublingual or topical. The compound for use provided herein may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example, an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compound may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The disclosure is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Synthesis of Compound 1

The synthesis of Compound 1 is disclosed in International Application No. PCT/US2022/021461, the entire content of which is incorporated herein by reference. This compound is referred to as Example 239 in PCT/US2022/021461 and was obtained as off-white solid (referred to herein as “Pattern A”). LCMS: m/z=453.1 (M+H⁺) ¹H NMR: (400 MHz, CHLOROFORM-d) δ=8.69-8.29 (m, 2H), 7.35 (br d, J=7.1 Hz, 1H), 7.08 (br d, J=7.1 Hz, 4H), 6.89-6.73 (m, 1H), 5.31 (br s, 2H), 5.00 (br s, 2H), 4.08 (br d, J=6.5 Hz, 2H), 3.09 (br s, 3H), 1.39 (br t, J=5.9 Hz, 3H). This compound was shown to have an IC₅₀ value of <1 uM against NLRP3.

### Analytical Methods for Polymorph Studies

Unless otherwise indicated, X-ray powder diffraction (XRPD) was performed on a Bruker D8 Advance diffractometer in reflection mode using collimated Cu Kα radiation operating at 40 kV, 40 mA. Scans were run from 2-40 degrees 2-theta with a step size of 0.02 degrees and a scan time of 0.3 second per step.

Differential Scanning calorimetry (DSC) was conducted on a TA Discovery 2500 with a Tzero pan and Tzero hermetic lid with a pin hole of 0.7 mm in diameter. DSC analysis was performed by ramping 10° C./min from 0 to 250° C.

Thermal gravimetric analysis (TGA) was conducted on Discovery 5500 or Q5000 with a start temperature at ambient conditions (below 35° C.) and a final temperature of 300° C. (or abort next segment if weight<80% (w/w)) with a heat 10° C./min.

Nuclear magnetic resonance (NMR) analysis was run on a Bruker Avance-AV 400M at a frequency of 400 MHz for eight scans.

Dynamic vapor sorption (DVS) was performed on an Intrinsic, Advantage or Adventure with an oven temperature of 25° C. using water as a solvent. The sample mass was about 5-10 mg using the following method:
Cycle: 40-0-95-0-40% RH
Stage Step: 10%
Equilibrium: 0.002 dm/dt (%/min)
Minimum dm/dt stability duration: 60 min
Maximum dm/dt stage time: 360 min.

### Example 1. Preparation of Crystal Forms

Compound 1 in free form shows polymorphic behaviors. In total, three crystalline forms were identified to be polymorphs of the free form including one hydrate (Pattern A) and two anhydrates (Pattern B and Pattern C). In addition, an amorphous form was identified in heat-cool DSC cycle and variable temperature XRPD experiments. HCl salt Pattern A, phosphate salt Pattern A, and sodium salt Pattern A were scaled up successfully. These scale-up batches are the same polymorphs as those of the screening samples.

### Preparation of Compound 1 Patterns A, B, and C

Pattern A is a monohydrate. It can be obtained in water and solvent water/systems by equilibration and anti-solvent addition. It can be also obtained in organic solvents by slow evaporation. Pattern A is of high crystallinity. It contains 1.0 equivalent (3.8% by weight) of water according to KF result. DSC shows a dehydration peak from about 8° C. with an enthalpy of about 114 J/g and a melting peak at Tₒₙₛₑₜ of 134.2° C. with an enthalpy of about 66 J/g. TGA shows about 3.8% weight loss at about 100° C. 1H-NMR shows no detectable residual solvent. After dehydration, Pattern A converted to anhydrate Pattern C. Pattern C was unstable and converted to Pattern A once exposure in ambient condition (˜23° C., ˜27% RH). Water activity experiments showed that Pattern A converted to Pattern B in water activity ranging from 0 to 0.4 at 5° C. and from 0 to 0.6 at 25° C.

Pattern B is an anhydrate. It was obtained from most of organic solvents by equilibration and slow cooling. It can be also obtained from acetonitrile/MTBE and THF/MTBE by anti-solvent addition. Pattern B is of high crystallinity. DSC shows a melting peak at Tₒₙₛₑₜ of 174.8° C. with an enthalpy of about 117 J/g. TGA shows about 0.4% weight loss at about 150° C. 1H-NMR shows no detectable residual solvent. Water activity experiments showed that Pattern B is the thermodynamically stable form in water activity ranging from 0 to 0.4 at 5° C. and from 0 to 0.6 at 25° C.

Pattern C is an anhydrate. It was obtained in variable temperature XRPD experiment by heating Pattern A to 100° C. under the nitrogen atmosphere. Pattern C is of high crystallinity. According to the DSC thermogram of Pattern A, Pattern C has a melting peak at Tₒₙₛₑₜ of 134.2° C. Pattern C is a metastable form. After exposure in ambient condition (23° C., 27% RH), Pattern C converted to Pattern A.

### Competitive Equilibration and Water Activity Experiments of Compound 1 Free Form

Relative stability of the hydrate Pattern A and the anhydrate Pattern B was investigated by competitive equilibration and water activity experiments at 5° C. and 25° C.

After equilibration for 5 days, hydrate Pattern A converted to anhydrate Pattern B in water activity ranging from 0 to 0.4 at 5° C. and from 0 to 0.6 at 25° C. In water activity ranging from 0.6 to 1.0 at 5° C. and from 0.8 to 1.0 at 25° C., an obtained sample remained as a physical mixture of Pattern A and Pattern B. It is presumed that the slow form transition kinetics was caused by the low solubility.

Based on above results, Pattern B is the thermodynamically stable form in water activity ranging from 0 to 0.4 at 5° C. and from 0 to 0.6 at 25° C. and is considered to be the optimal polymorphic form of free form.

### Evaluation of Pattern B

### Bulk Stability

Bulk stability of free form Pattern B was evaluated at 25° C./92.5% RH in an open container, at 40° C./75% RH in an open container and at 60° C. in a tight container over 1 week.

Free form Pattern B was physically and chemically stable under these conditions. HPLC showed no obvious degradation. XRPD showed no form change.

### Solubility

Solubility of BAL-0748 free form Pattern B was investigated in pH 1.2 HCl buffer, pH 4.5 50 mM acetate buffer, pH 6.8 50 mM phosphate buffer, water, pH 1.6 FaSSGF, pH 6.5 FaSSIF-v1, pH 5.0 FeSSIF-v1 and 2% HPMC+1% Pluronic F68 in pH 4.5 50 mM acetate buffer at 37° C. for 2 h and 24 h. Residual solids after the solubility test were analyzed by XRPD.

Free form Pattern B showed good solubility of 0.1-0.6 mg/ml in pH 1.2 HCl buffer, pH 1.6 FaSSGF and pH 5.0 FeSSIF-v1. In other media, the free form Pattern B showed poor solubility of 2-80 μg/mL at 2 h and 24 h.

After the solubility test, free form Pattern B showed no form change or partially converted to free form Pattern A in most of media. However, in pH 1.2 HCl buffer and pH 1.6 FaSSGF, salt formation occurred. Mono-HCl salt Pattern A and hemi-HCl salt Pattern A were obtained in pH 1.2 HCl buffer and pH 1.6 FaSSGF, respectively.

### Hygroscopicity

Hygroscopicity of free form Pattern B was evaluated by dynamic vapor sorption (DVS) test at 25° C. Free form Pattern B was slightly hygroscopic. It absorbed about 0.8% water at 80% RH at 25° C. After the DVS test, obtained sample was still Pattern B.

### Preparation of Compound 1 HCl Salt Pattern A

HCl salt Pattern A was prepared using the procedure below.

300.3 mg of the Compound 1 free form Pattern A was weighed into a 20 mL glass vial. 4 mL of IPA was added into the vial at 50° C. Then 0.57 mL of 1.2N HCl aqueous solution (˜1.05 equivalent by molar ratio) was added into above solution. After stirring at 50° C. for about 2 min, a suspension was obtained. (suspension)

About 10 mg of the HCl salt Pattern A seeds were added into above solution (suspension). After stirring at 50° C. for about 2 hours, the solution was cooled to 25° C. by natural cooling (suspension). The suspension was kept stirring at 25° C. for about 5 days.

Solids were collected by centrifugation filtration through a 0.45 μm nylon membrane filter by centrifugation and then dried at 50° C. under vacuum for about 2 hours. 295 mg of the HCl salt Pattern A was obtained as a white solid in 90% yield. Characterization of Compound 1 HCl salt Pattern A is reported in Table 9 below.

### Preparation of Compound 1 Phosphate Salt Pattern A

Phosphate salt Pattern A was prepared using the procedure below.

300.0 mg of the BAL-0748 free form Pattern A was weighed into a 20 mL glass vial. 7 mL of IPA was added into the vial at 50° C. Then 0.48 mL of diluted phosphoric acid solution (˜1.05 equivalent by molar ratio, 0.1 mL of phosphoric acid was diluted with 0.9 mL of IPA) was added into above solution. A suspension was obtained. About 10 mg of the phosphate salt Pattern A seeds were added into above solution (suspension). After stirring at 50° C. for about 2 hours, the solution was cooled to 25° C. by natural cooling. The suspension was kept stirring at 25° C. for about 5 days.

Solids were collected by centrifugation filtration and then dried at 50° C. under vacuum for about 2 hours. 336 mg of the phosphate salt Pattern A was obtained as a white solid in 91% yield. Characterization of Compound 1 phosphate salt Pattern A is reported in Table 9 below.

### Preparation of Compound 1 Sodium Salt Pattern A

Sodium salt Pattern A was prepared using the procedure below.

300.3 mg of the Compound 1 free form Pattern A and about 29 mg of NaOH (˜1.05 equivalent by molar ratio) were weighed into a 20 mL glass vial. 3 mL of IPA was added into the vial at 50° C. A suspension was obtained. (almost clear solution). About 10 mg of sodium salt Pattern A seeds were added into above solution. After stirring at 50° C. for about 10 min, the solution gradually converted into a suspension. (almost clear solution→suspension).

After stirring at 50° C. for about 2 hours, the solution was cooled to 25° C. by natural cooling (suspension). The suspension was kept stirring at 25° C. for about 5 days. Solids were collected by centrifugation filtration and then dried at 50° C. under vacuum for about 2 hours. 283 mg of the sodium salt Pattern A was obtained as a white solid in 89% yield. Characterization of Compound 1 sodium salt Pattern A is reported in Table 9.

### Example 2. Salt/Cocrystal Screening

Compound 1 is a small molecule with molecular weight of 452.46 g/mol. As predicted by Marvin Sketch v21.3, the compound contains 1 basic pKa of 5.16, 1 acidic pKa of 9.29 as well as 2H-bond donors and 5H-bond acceptors. Compound 1 free form Pattern A was used as starting material in this salt/cocrystal screening study. Pattern A is a monohydrate.

Based on the pKa(s) of Compound 1, seven Class I acids, two Class II acids, and two Class I bases were selected as salt forming agents: hydrochloric acid, sulfuric acid, phosphoric acid, L-aspartic acid, maleic acid, L-glutamic acid, fumaric acid, methanesulfonic acid, p-toluenesulfonic acid, sodium hydroxide, and potassium hydroxide. Based on in silico cocrystal prediction results and considering H-bond synthon preference of Compound 1, two cocrystal formers (coformers) were also selected to pursue potential cocrystals: choline and oxalic acid. IPA, EA, and acetonitrile/water (95:5, v/v) were used as screening solvents. 1.0 equivalent or 0.5 equivalent of selected counter ions/coformers were applied in the screening. Slurry equilibration, cooling and anti-solvent addition were used as crystallization methods. In total, 53 screening experiments were conducted.

According to the salt/cocrystal screening results (Tables 6-8), eighteen salts/cocrystals and their polymorphs were identified, including HCl salt Pattern A, hemi-sulfate salt Pattern A, hemi-sulfate salt Pattern B, mono-sulfate salt Pattern A, phosphate salt Pattern A, maleate salt Pattern A, hemi-fumarate salt Pattern A, hemi-fumarate salt Pattern B, mono-fumarate salt Pattern A, mesylate salt Pattern A, mesylate salt Pattern B, mesylate salt Pattern C, p-tosylate salt Pattern A, p-tosylate salt Pattern B, oxalate Pattern A, sodium salt Pattern A, potassium salt Pattern A and potassium salt Pattern B. All these salt/cocrystal hits were further characterized by DSC, TGA, ¹H-NMR, IC, KF, HPLC and PLM.

About 50 mg of the Compound 1 free form Pattern A and 0.5 or 1.0 equiv. of acid or base were added into a screening solvent in a 2 mL glass vial. Obtained mixtures were stirred at 50° C. for 2 hours and then at 25° C. for about 3 days.

TABLE 6
Slurry equilibration

| | | | C |
|---|---|---|---|
| Counter ions/ | A | B | ACN/water (95:5, |
| coformers | IPA | EA | v/v) |
| N/A (free form | Free form | Free form Pattern B | Free form Pattern A |
| only) | Pattern B | | |
| Hydrochloric | HCl salt Pattern | HCl salt Pattern A with extra | HCl salt Pattern A |
| acid (1.0 equiv.) | A | peaks at 2 theta 13.0° and | |
| | | 15.5°; | |
| | | Thermal events: | |
| | | Desolvation from about 73° C. | |
| | | Endothermic onset: 145.7° C. | |
| | | Melting Tₒₙₛₑₜ @ 202.3° C. | |
| | | Weight loss: ~1.2% @ 130° C. | |
| | | ¹HNMR: 1.1% (0.06 equiv.) EA | |
| | | IC: free form:HCl = 1: 0.74 | |
| Sulfuric acid (1.0 | Hemi-sulfate salt | Mono-sulfate salt Pattern A | Hemi-sulfate salt |
| equiv.) | Pattern A | | Pattern B |
| Phosphoric acid | Phosphate salt | Phosphate salt Pattern A | Phosphate salt |
| (1.0 equiv.) | Pattern A | | Pattern A |
| L-Aspartic acid | Physical mixture | Free form Pattern B | Free form Pattern A |
| (1.0 equiv.) | of free form | | |
| | Pattern B and L- | | |
| | aspartic acid | | |
| Maleic acid (1.0 | Maleate salt | Maleate salt Pattern A | Maleate salt |
| equiv.) | Pattern A | | Pattern A |
| L-Glutamic acid | Free form | Free form Pattern B + L- | Free form Pattern A + |
| (1.0 equiv.) | Pattern B + L- | glutamic acid | L-glutamic acid |
| | glutamic acid | | |
| Fumaric acid | Hemi-fumarate | Hemi-fumarate salt Pattern A + | Mono-fumarate salt |
| (1.0 equiv.) | salt Pattern A | fumaric acid+ extra peaks at 2 | Pattern A |
| | | theta 3.5°, 6.9°, 10.3°, 13.9° | |
| Fumaric acid | Hemi-fumarate | Hemi-fumarate salt Pattern A + | Hemi-fumarate salt |
| (0.5 equiv.) | salt Pattern A | mono-fumarate salt Pattern A + | Pattern B |
| | | hemi-fumarate salt Pattern B | |
| | | with one extra peak at 2 theta | |
| | | 6.9° | |
| Methanesulfonic | Clear solution | Mesylate salt Pattern A | Clear solution |
| acid (1.0 equiv.) | | | |
| p- | p-tosylate salt | p-tosylate salt Pattern B | p-tosylate salt |
| Tolunenesulfonic | Pattern A | | Pattern B |
| acid (1.0 | | | |
| equiv.) | | | |
| NaOH (1.0 | Sodium salt | Sodium salt Pattern A with one | Sodium salt Pattern |
| equiv.) | Pattern A | extra peak at 2 theta 8.8° | A with one extra |
| | | | peak at 2 theta 8.8° |
| KOH (1.0 equiv.) | Clear solution | Potassium salt Pattern A | Potassium salt |
| | | | Pattern B |
| Choline (1.0 | Clear solution | Free form Pattern A | Clear solution |
| equiv.) | | | |
| Oxalic acid (1.0 | Oxalate Pattern | Oxalate Pattern A | Oxalate Pattern A |
| equiv.) | A | | |

Clear solutions obtained in slurry equilibration experiments were cooled to 5° C. to precipitate solids. After stirring at 5° C. for about 3 days, suspensions obtained after cooling were filtered through a 0.45 μm nylon membrane filter by centrifugation at 14,000 rpm. After being dried at 50° C. under vacuum for 2 h, solids were analyzed by XRPD.

TABLE 7
Cooling

| | | | C |
|---|---|---|---|
| Counter ions/ | A | B | ACN/water (95:5, |
| coformers | IPA | EA | v/v) |
| Methanesulfonic | Mesylate salt | // | Mesylate salt |
| acid (1.0 equiv.) | Pattern B | | Pattern C |
| KOH (1.0 equiv.) | Hazy suspension | // | // |
| Choline (1.0 equiv.) | Clear solution | // | Hazy suspension |

Clear solutions or hazy suspensions obtained from cooling experiments were further treated by addition of anti-solvent. Suspensions obtained after anti-solvent addition were filtered through a 0.45 μm nylon membrane filter by centrifugation at 14,000 rpm. After being dried at 50° C. under vacuum for 2 h, solids were analyzed by XRPD.

TABLE 8
Anti-solvent addition

| Counter ions/ | Good | Anti- | |
|---|---|---|---|
| coformers | solvent (mL) | solvent (μL) | Comments |
| KOH (1.0 | IPA (0.6) | Heptane (2.4) | Similar to potassium |
| equiv.) | | | salt Pattern B |
| Choline (1.0 | IPA (0.5) | MTBE (2.0) | Clear solution |
| equiv.) | | | |
| Choline (1.0 | ACN/water | MTBE (2.4) | Gel-like sample |
| equiv.) | (95:5, v/v) (0.6) | | |

Among these salt/cocrystal hits, the HCl salt Pattern A, the phosphate salt Pattern A and the sodium salt Pattern A showed good properties including high purity, high crystallinity, high melting point, reasonable stoichiometry, and good counter ion safety. Therefore, they were selected as salt/cocrystal candidates for scale-up and full evaluation in comparison with the free from Pattern A in terms of bulk stability, solubility, hygroscopicity and morphic properties.

Free form Pattern A, monohydrate, HCl salt Pattern A, anhydrate, phosphate salt Pattern A, anhydrate, and sodium salt Pattern A, anhydrate were scaled up and fully evaluated. The results are summarized below.

TABLE 9
Chemical and physicochemical properties

| Physical Form | | | |
|---|---|---|---|
| Free form | | Phosphate salt | Sodium salt |
| Pattern A, | HCl salt Pattern | Pattern A, | Pattern A, |
| monohydrate | A, anhydrate | anhydrate | anhydrate |

Initial chemical purity by HPLC

| | | | | |
|---|---|---|---|---|
| Purity [area %] | 97.8% | 98.8% | 98.1% | 98.8% |

Stoichiometry by IC

| | | | | |
|---|---|---|---|---|
| Free form: | N/A | Free form:HCl = | Free form: | Free form: |
| counter ion | | 1:0.94 | H₃PO₄ = 1:1.06 | NaOH = 1:1.06 |

Residual solvent(s) by ¹H-NMR

| | | | | |
|---|---|---|---|---|
| Weight (%) | No detectable | 0.6% (0.05 | 0.2% (0.02 | 0.4% (0.03 |
| | residual solvent | equiv.) IPA | equiv.) IPA | equiv.) IPA |
| | | residue | residue | residue |

Water content by Karl Fischer (for hydrate only)

| | | | | |
|---|---|---|---|---|
| Weight (%) | 3.8% water by | // | // | // |
| | weight (equal to | | | |
| | 0.99 equiv.) | | | |

Crystallinity by XRPD

| | | | | |
|---|---|---|---|---|
| High/medium/low | High crystallinity, | High crystallinity, | High crystallinity, | High crystallinity, |
| | free form Pattern | HCl salt pattern A | phosphate salt | sodium salt |
| | A (FIG. 4) | (FIG. 7) | Pattern A | Pattern A (FIG. |
| | | | (FIG. 10) | 13) |

DSC, heating rate [10° C./min]

| | | | | |
|---|---|---|---|---|
| Thermal events | Dehydration from | Melting Tₒₙₛₑₜ @ | Endothermic | No melting |
| (C) | about 8° C.; | 211.2° C. | Tₒₙₛₑₜ @153.0° C. | appears before |
| | Melting Tₒₙₛₑₜ @ | (FIG. 8) | Melting Tₒₙₛₑₜ @ | decomposition. |
| | 134.2° C. | | 221.1° C. | (FIG. 14) |
| | (FIG. 5) | | (FIG. 11) | |
| Enthalpy (J/g) | Dehydration | Decomposition | Endothermic | N/A |
| | enthalpy: 114 J/g; | occurs upon | enthalpy: 2 J/g | |
| | Melting enthalpy: | melting. | Decomposition | |
| | 66 J/g | | occurs upon | |
| | | | melting. | |

Themogravimetry, heating rate [10° C./min]

| | | | | |
|---|---|---|---|---|
| Weight loss in (% | 3.8% @ 100° C. | 1.0% @ 180° C. | 0.5% @ 195° C. | About 1.5% @ |
| @ ° C.) | (FIG. 6) | (FIG. 9) | (FIG. 11) | 250° C. (FIG. |
| | | | | 15) |

Free form Pattern A is a monohydrate of high crystallinity. DSC shows a dehydration peak from about 8° C. with an enthalpy of about 114 J/g and a melting peak at Tₒₙₛₑₜ Of 134.2° C. with an enthalpy of about 66 J/g. TGA shows about 3.8% weight loss at about 100° C. HPLC shows 97.8% chemical purity. KF shows 3.8% water by weight, equivalent to 1.0 water molecule. 1H-NMR shows no detectable residual solvent.

HCl salt Pattern A is an anhydrate of high crystallinity. DSC shows a melting peak at Tₒₙₛₑₜ of 211.2° C. Decomposition occurs upon melting. TGA shows about 1.0% weight loss at 180° C. HPLC shows 98.8% chemical purity. IC shows stoichiometric ratio of free form to HCl is 1:0.94. ¹H-NMR shows 0.6% (equal to 0.05 equiv. by molar ratio) residual IPA by weight.

Phosphate salt Pattern A is an anhydrate of high crystallinity. DSC shows an endothermic peak at Tₒₙₛₑₜ of 153.0° C. with an enthalpy of 2 J/g and a melting peak at Tₒₙₛₑₜ Of 221.1° C. Decomposition occurs upon melting. TGA shows about 0.5% weight loss at 195° C. HPLC shows 98.1% chemical purity. IC shows stoichiometric ratio of free form to H₃PO₄ is 1:1.06. ¹H-NMR shows 0.2% (equal to 0.02 equiv. by molar ratio) residual IPA by weight.

Sodium salt Pattern A is an anhydrate of high crystallinity. DSC shows no melting peak appears before decomposition. TGA shows about 1.5% weight loss at 250° C. HPLC shows 98.8% chemical purity. IC shows stoichiometric ratio of free form to NaOH is 1:1.06. ¹H-NMR shows 0.4% (equal to 0.03 equiv. by molar ratio) residual IPA by weight.

### Example 3. Bulk Stability

The free form Pattern A, the HCl salt Pattern A, and the phosphate salt Pattern A were chemically and physically stable under these conditions over 1 week. XRPD showed no form change. HPLC showed no obvious degradation.

The sodium salt Pattern A was chemically stable but physically unstable. HPLC showed no obvious degradation. It showed no form change at 60° C. but dissociated to free form Pattern A at 25° C./92.5% RH and 40° C./75% RH after 1 week. This result suggested that dissociation of sodium salt was high humidity relevant.

The results of these studies are summarized in Table 10.

TABLE 10
Stability: purity and appearance (color, CL)

| | Physical Form | | | | | | | |
|---|---|---|---|---|---|---|---|---|
| | Free form | | | | Phosphate salt | | Sodium salt | |
| | Pattern A, | | HCl salt Pattern | | Pattern A, | | Pattern A, | |
| | monohydrate | | A, anhydrate | | anhydrate | | anhydrate | |
| Initial purity | 97.8% | | 98.8% | | 98.1% | | 98.8% | |
| by HPLC | | | | | | | | |
| Initial color | White | | White | | White | | White | |
| Stoichiometry | N/A | | Free form:HCl = 1: | | Free form: | | Free form: | |
| by IC | | | 0.94 | | H₃PO₄ = 1:1.06 | | NaOH = 1:1.06 | |
| | Purity | CL | Purity | CL | Purity | CL | Purity | CL |

Solid state, 25° C./92.5% RH, open, 1 week

| | | | | | | | | |
|---|---|---|---|---|---|---|---|---|
| Bulk (HPLC) | 97.9% | A | 99.1% | A | 98.2% | A | 98.7% | A |

| | | | | |
|---|---|---|---|---|
| Bulk (XRPD) | No form | No form change | No form change | Free form |
| | change | | | Pattern A with |
| | | | | two extra peaks |
| | | | | at 2 theta 17.0° |
| | | | | and 38.0° |
| Stoichiometry | N/A | Free form:HCl = 1: | // | // |
| by IC | | 0.97 | | |

Solid state, 40° C./75% RH, open, 1 week

| | | | | | | | | |
|---|---|---|---|---|---|---|---|---|
| Bulk (HPLC) | 97.7% | A | 98.9% | A | 98.2% | A | 98.7% | A |

| | | | | |
|---|---|---|---|---|
| Bulk (XRPD) | No form | No form change | No form change | Free form |
| | change | | | Pattern A with |
| | | | | two extra peaks |
| | | | | at 2 theta 17.0° |
| | | | | and 38.0° |
| Stoichiometry | N/A | Free form:HCl = 1: | // | // |
| by IC | | 0.99 | | |

Solid state, 60° C., closed, 1 week

| | | | | | | | | |
|---|---|---|---|---|---|---|---|---|
| Bulk (HPLC) | 97.8% | A | 98.9% | A | 98.2% | A | 98.7% | A |

| | | | | |
|---|---|---|---|---|
| Bulk (XRPD) | No form | No form change | No form change | No form change |
| | change | | | |
| Stoichiometry | N/A | Free form:HCl = 1: | // | // |
| by IC | | 0.97 | | |

Explanation
A: no change of color;
B: slight discoloration
C: medium discoloration;
D: strong discoloration
“N/A”: Not applicable
“//”: Not carry out.

### Example 4. Solubility

Solubility of the free form Pattern A, the HCl salt Pattern A, the phosphate salt Pattern A, and the sodium salt Pattern A was measured in pH 1.2 HCl buffer, pH 4.5 acetate buffer (50 mM), pH 6.8 phosphate buffer (50 mM), water, pH 1.6 FaSSGF, pH 6.5 FaSSIF-v1, pH 5.0 FeSSIF-v1, and 2% HPMC+1% Pluronic F68 in pH 4.5 acetate buffer (50 mM) at 37° C. for 2 hours and 24 hours. Residual solids after the solubility test at 24 h were analyzed by XRPD.

In pure water, the HCl salt Pattern A and the phosphate salt Pattern A showed obviously higher solubility compared with the free form Pattern A, which should be caused by pH shift.

In pH 5.0 FeSSIF-v1, the HCl salt Pattern A showed 1-fold higher solubility compared with the free form Pattern A at 2 h. At 24 h, the three salts showed comparable solubility with the free form Pattern A.

In pH 1.2 HCl buffer, pH 4.5 acetate buffer, pH 6.8 phosphate buffer and pH 6.5 FaSSIF-v1, the three salts showed comparable solubility with the free form Pattern A.

In 2% HPMC+1% Pluronic F68 in acetate buffer (pH 4.5), the sodium salt Pattern A showed obviously improved solubility while the HCl salt Pattern A and the phosphate salt Pattern A showed comparable solubility with the free form Pattern A.

After the solubility test, the three salt candidates partially or completely dissociated to the free form Pattern A in all the media except in pH 1.2 HCl buffer and in pH 1.6 FaSSGF. Mono-HCl salt Pattern A or hemi-HCl salt Pattern A was obtained in pH 1.2 HCl buffer or pH 1.6 FaSSGF.

10.4 mg of the free form Pattern A, 10.0 mg of the free form Pattern B, 10.8 mg of the HCl salt Pattern A, 12.2 mg of the phosphate salt Pattern A, or 10.5 mg of the sodium salt Pattern A was weighed into a 20 mL glass vial, respectively. 5 mL of solubility medium was added. The salt amount used was equivalent to 10 mg anhydrous free form. Obtained suspensions were stirred at 37° C. at 400 rpm and sampled at 2 hours and at 24 hours. The samples were centrifuged at 37° C. at 14,000 rpm for 5 min. Supernatants were analyzed by HPLC and PH meter for solubility and pH value, respectively. Residual solids (wet cakes) from the 24 hours samples were also characterized by XRPD to determine physical form. The results of this study are shown in Table 11.

TABLE 11
Solubility
Solubility at 37° C., target concentration 2 mg/mL (in free form), equilibration for 24 hours, LOQ: 0.25 mg/mL

| | Physical Form | | | | | | | | | | | |
|---|---|---|---|---|---|---|---|---|---|---|---|---|
| | Free form Pattern A, | | | HCl salt Pattern A, | | | Phosphate salt Pattern A, | | | Sodium salt Pattern A, | | |
| | monohydrate | | | anhydrate | | | anhydrate | | | anhydrate | | |
| | Solubility | | | Solubility | | | Solubility | | | Solubility | | |
| | (μg/mL) | | XRPD of | (μg/mL) | | XRPD of | (μg/mL) | | XRPD of | (μg/mL) | | XRPD of |
| Solubility | | 24 h | residual | | 24 h | residual | | 24 h | residual | | 24 h | residual |
| media | 2 h | (pH) | solid | 2 h | (pH) | solid | 2 h | (pH) | solid | 2 h | (pH) | solid |
| pH 1.2 | 425.4 | 401.9 | HCl salt | 361.6 | 276.8 | HCl salt | 402.0 | 390.0 | HCl salt | 608.5 | 510.7 | HCl salt |
| HCl | | (1.2) | Pattern | | (1.2) | Pattern | | (1.2) | Pattern A | | (1.3) | Pattern |
| buffer | | | A with a | | | A with a | | | with a | | | A with a |
| | | | little bit | | | little bit | | | little bit | | | little bit |
| | | | difference | | | difference | | | difference | | | difference |
| | | | at 2 | | | at 2 | | | at 2 | | | at 2 |
| | | | theta | | | theta | | | theta | | | theta |
| | | | 28.3° | | | 28.3° | | | 28.3° | | | 28.3° |
| pH 4.5 | 14.4 | 13.7 | Free | 14.4 | 10.0 | Free | 9.4 | 11.8 | Free form | 9.3 | 7.4 | Free |
| acetate | | (4.5) | form | | (4.3) | form | | (4.4) | Pattern A + | | (4.6) | form |
| buffer | | | Pattern | | | Pattern | | | Phosphate | | | Pattern |
| (50 mM) | | | A | | | A with | | | salt | | | A with |
| | | | | | | one | | | Pattern A | | | one |
| | | | | | | extra | | | with one | | | extra |
| | | | | | | peak at | | | extra | | | peak at |
| | | | | | | 2theta | | | peak at | | | 2theta |
| | | | | | | 8.9° | | | 2theta | | | 8.9° |
| | | | | | | | | | 8.9° | | | |
| pH 6.8 | 4.7 | 4.3 | Free | 3.3 | 2.3 | Free | 2.4 | 2.9 | Free form | 3.1 | 2.8 | Free |
| phosphate | | (6.8) | form | | (6.7) | form | | (6.7) | Pattern A + | | (7.0) | form |
| buffer | | | Pattern | | | Pattern | | | Phosphate | | | Pattern |
| (50 mM) | | | A with | | | A with | | | salt | | | A |
| | | | two | | | two | | | Pattern A | | | |
| | | | extra | | | extra | | | | | | |
| | | | peaks at | | | peaks at | | | | | | |
| | | | 19.1° | | | 2theta | | | | | | |
| | | | and | | | 19.1° and | | | | | | |
| | | | 38.8° | | | 38.8° | | | | | | |
| Water | 5.6 | 4.6 | Free | 568.4 | 504.3 | Free | 383.6 | 442.2 | Free form | 7.7 | 8.2 | Free |
| | | (6.3) | form | | (2.5) | form | | (2.6) | Pattern A | | (11.1) | form |
| | | | Pattern | | | Pattern | | | | | | Pattern |
| | | | A | | | A | | | | | | A |
| FaSSGF, | 745.9 | 615.7 | Should | 555.7 | 522.2 | HCl salt | 710.2 | 652.6 | HCl salt | 664.5 | 629.6 | Should |
| pH 1.6 | | (1.6) | be a | | (1.5) | Pattern | | (1.6) | Pattern A | | (1.8) | be a |
| | | | hemi- | | | A with a | | | with a | | | hemi- |
| | | | HCl salt | | | little bit | | | little bit | | | HCl salt |
| | | | Pattern | | | difference | | | difference | | | Pattern |
| | | | A + HCl | | | at 2 | | | at 2 | | | A |
| | | | salt | | | theta | | | theta | | | |
| | | | Pattern | | | 28.3° | | | 28.3° | | | |
| | | | A | | | | | | | | | |
| FaSSIF- | 14.5 | 17.7 | Free | 11.8 | 8.4 | Free | 8.4 | 13.7 | Free form | 10.2 | 13.1 | Free |
| v1, pH | | (6.5) | form | | (6.3) | form | | (6.2) | Pattern A + | | (6.7) | form |
| 6.5 | | | Pattern | | | Pattern | | | Phosphate | | | Pattern |
| | | | A | | | A | | | salt | | | A |
| | | | | | | | | | Pattern A | | | |
| FeSSIF- | 142.1 | 168.5 | Free | 238.5 | 150.9 | Free | 140.0 | 159.1 | Free form | 144.4 | 149.3 | Free |
| v1, pH | | (5.0) | form | | (4.9) | form | | (4.9) | Pattern A | | (5.1) | form |
| 5.0 | | | Pattern | | | Pattern | | | | | | Pattern |
| | | | A | | | A with | | | | | | A |
| | | | | | | one | | | | | | |
| | | | | | | extra | | | | | | |
| | | | | | | peak at | | | | | | |
| | | | | | | 2theta | | | | | | |
| | | | | | | 8.9° | | | | | | |
| 2% | 24.4 | 34.3 | Free | 29.5 | 26.7 | Free | 16.4 | 17.9 | Free form | 273.3 | 260.2 | Free |
| HPMC + | | (4.6) | form | | (4.4) | form | | (4.5) | Pattern A + | | (4.7) | form |
| 1% | | | Pattern | | | Pattern | | | phosphate | | | Pattern |
| Pluronic | | | A | | | A | | | salt | | | A |
| F68 in | | | | | | | | | Pattern A | | | |
| pH 4.5 | | | | | | | | | | | | |
| acetate | | | | | | | | | | | | |
| buffer | | | | | | | | | | | | |
| (50 mM) | | | | | | | | | | | | |

### Example 5. Hygroscopicity

Hygroscopicity of the free form Pattern A, the HCl salt Pattern A, the phosphate salt Pattern A, and the sodium salt Pattern A was evaluated by dynamic vapor sorption (DVS) test at 25° C.

The free form Pattern A, the HCl salt Pattern A and the phosphate salt Pattern A were slightly hygroscopic and showed no form change after the DVS test at 25° C.

The sodium salt Pattern A was moderately hygroscopic. It absorbed 2.7% water at 80% RH at 25° C. After the DVS test, it showed no form change. These results are shown in Table 12.

TABLE 12
Hygroscopicity
Hygroscopicity by DVS at 25° C.
Physical Form

| | | | | | | | | | Phosphate salt | | | | Sodium salt | | | |
|---|---|---|---|---|---|---|---|---|---|---|---|---|---|---|---|---|
| Relative | Free form Pattern | | | | HCl salt Pattern A, | | | | Pattern A, | | | | Pattern A, | | | |
| humidity | A, monohydrate | | | | anhydrate | | | | anhydrate | | | | anhydrate | | | |
| at | Sorp. | Desorp. | Sorp. | Desorp. | Desorp. | Sorp. | Desorp. | Sorp. | Desorp. | Sorp. | Desorp. | Sorp. | Desorp. | Sorp. | Desorp. | Sorp. |
| 25° C. | (%) | (%) | (%) | (%) | (%) | (%) | (%) | (%) | (%) | (%) | (%) | (%) | (%) | (%) | (%) | (%) |
| 0% | // | 0.01 | 0.01 | // | 0.51 | 0.51 | 0.15 | 0.15 | 0.03 | 0.03 | 0.00 | 0.00 | 0.05 | 0.05 | 0.00 | 0.00 |
| 10% | // | 3.59 | 0.09 | // | 0.61 | 0.61 | 0.24 | 0.20 | 0.12 | 0.11 | 0.09 | 0.09 | 0.20 | 0.17 | 0.18 | 0.14 |
| 20% | // | 3.74 | 3.45 | // | 0.65 | 0.68 | 0.28 | 0.24 | 0.16 | 0.15 | 0.14 | 0.13 | 0.28 | 0.23 | 0.25 | 0.21 |
| 30% | // | 3.79 | 3.78 | // | 0.69 | 0.78 | 0.35 | 0.27 | 0.20 | 0.18 | 0.18 | 0.17 | 0.35 | 0.30 | 0.33 | 0.28 |
| 40% | 3.87 | 3.83 | 3.82 | 3.83 | 0.72 | 0.75 | 0.42 | 0.29 | 0.24 | 0.23 | 0.23 | 0.21 | 0.45 | 0.38 | 0.94 | 0.38 |
| 50% | 3.90 | 3.93 | 3.87 | 3.92 | // | 0.80 | 0.54 | // | // | 0.27 | 0.29 | // | // | 0.49 | 1.69 | // |
| 60% | 3.94 | 4.00 | 3.93 | 3.99 | // | 0.85 | 0.68 | // | // | 0.32 | 0.35 | // | // | 0.94 | 2.23 | // |
| 70% | 4.01 | 4.07 | 4.00 | 4.06 | // | 0.96 | 0.84 | // | // | 0.37 | 0.41 | // | // | 1.87 | 2.55 | // |
| 80% | 4.12 | 4.17 | 4.11 | 4.15 | // | 1.00 | 1.07 | // | // | 0.45 | 0.48 | // | // | 2.71 | 3.05 | // |
| 90% | 4.33 | 4.35 | 4.30 | 4.33 | // | 1.37 | 1.54 | // | // | 0.61 | 0.63 | // | // | 4.13 | 4.35 | // |
| 95% | 4.61 | 4.61 | 4.58 | 4.58 | // | 2.20 | 2.20 | // | // | 0.82 | 0.82 | // | // | 6.60 | 6.60 | // |
| Hygroscopicity | Slightly hygroscopic | | | | Slightly hygroscopic | | | | Slightly | | | | Moderately | | | |
| | It absorbed 0.3% | | | | It absorbed 1.0% | | | | hygroscopic | | | | hygroscopic | | | |
| | water from 40% RH | | | | water at 80% RH. | | | | It absorbed 0.5% | | | | It absorbed 2.7% | | | |
| | to 80% RH. | | | | | | | | water at 80% RH. | | | | water at 80% RH. | | | |
| XRPD | No form change | | | | No form change | | | | No form change | | | | No form change | | | |
| after | | | | | | | | | | | | | | | | |
| DVS | | | | | | | | | | | | | | | | |
| test | | | | | | | | | | | | | | | | |
| Stoichiometric | N/A | | | | Free form: HCl = | | | | // | | | | // | | | |
| ratio | | | | | 1:0.97 | | | | | | | | | | | |
| after | | | | | | | | | | | | | | | | |
| DVS | | | | | | | | | | | | | | | | |
| test | | | | | | | | | | | | | | | | |

Explanation “//” Not carried out.
“N/A”: Not applicable
Non-hygroscopic water update <0.2%
Slightly hygroscopic water update ≥0.2% but <2%
Moderately hygroscopic water uptake ≥2% but <15%
Very hygroscopic water uptake ≥15%
The criteria are modified from the European Pharmacopeia criteria about hygroscopicity.

### Example 6. Polymorphism

In this salt screening, one polymorph of the HCl salt (Pattern A) was identified; one polymorph of the phosphate salt (Pattern A) was identified; one polymorph of the sodium salt (Pattern A) was identified. In polymorph screening, three polymorphs of the free form (Pattern A, B, and C) were obtained.

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A crystalline form of 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide, or a pharmaceutically acceptable salt or hydrate thereof.

2. The crystalline form of claim 1, wherein the crystalline form is a monohydrate.

3. The crystalline form of claim 1, wherein the crystalline form is an anhydrate.

4. The crystalline form of claim 1, wherein the crystalline form is a pharmaceutically acceptable salt.

5. The crystalline form of claim 4, wherein the pharmaceutically acceptable salt is selected from the group consisting of hydrochloric acid salt, phosphoric acid salt, and sodium salt.

6. The crystalline form of claim 3, wherein the crystalline form is characterized by an XRPD diffractogram having peaks expressed in degrees-2-theta at angles (±0.2 degrees) of 7.2, 20.4, and 20.6.

7. The crystalline form of claim 3, wherein the crystalline form is characterized by an XRPD diffractogram having peaks expressed in degrees-2-theta at angles (±0.2 degrees) of 7.2, 20.4, 20.6, and 21.6.

8-9. (canceled)

10. The crystalline form of claim 3 having a DSC thermogram characterized by an endotherm with an onset temperature of 177.3° C.

11. The crystalline form of claim 2, wherein the crystalline form is characterized by an XRPD diffractogram having peaks expressed in degrees-2-theta at angles (±0.2 degrees) of 6.5, 12.9, and 16.1.

12. The crystalline form of claim 2, wherein the crystalline form is characterized by an XRPD diffractogram having peaks expressed in degrees-2-theta at angles (±0.2 degrees) of 6.5, 11.2, 12.9, and 16.1.

13. (canceled)

14. The crystalline form of claim 2 having a DSC thermogram characterized by an endotherm with an onset temperature of 139.6° C.

15. The crystalline form of claim 1, wherein the crystalline form is 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide hydrochloric acid salt.

16. The crystalline form of claim 15, wherein the crystalline form is characterized by an XRPD diffractogram having peaks expressed in degrees-2-theta at angles (±0.2 degrees) of 12.3, 18.1, and 18.2

17. The crystalline form of claim 15, wherein the crystalline form is characterized by an XRPD diffractogram having peaks expressed in degrees-2-theta at angles (±0.2 degrees) of 12.3, 12.8, 18.1, 18.2, and 28.7.

18. (canceled)

19. The crystalline form of claim 15 having a DSC thermogram characterized by an endotherm with an onset temperature of 218.3° C.

20. The crystalline form of claim 1, wherein the crystalline form is 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide phosphoric acid salt.

21. The crystalline form of claim 20, wherein the crystalline form is characterized by an XRPD diffractogram having peaks expressed in degrees-2-theta at angles (±0.2 degrees) of 3.1, 9.2, and 16.4.

22. The crystalline form of claim 20, wherein the crystalline form is characterized by an XRPD diffractogram having peaks expressed in degrees-2-theta at angles (±0.2 degrees) of 3.1, 9.2, 11.7, 13.9, 15.4, 16.4, and 24.7.

23. (canceled)

24. The crystalline form of claim 20 having a DSC thermogram characterized by an endotherm with an onset temperature of 223.9° C.

25. The crystalline form of claim 1, wherein the crystalline form is 2-ethoxy-3′,5′-difluoro-N-((4-(hydroxymethyl)-1H-pyrazolo[4,3-c]pyridin-7-yl)methyl)-N-methyl-[1,1′-biphenyl]-4-carboxamide sodium salt.

26. The crystalline form of claim 25, wherein the crystalline form is characterized by an XRPD diffractogram having peaks expressed in degrees-2-theta at angles (±0.2 degrees) of 10.3, 21.1, and 25.8.

27. The crystalline form of claim 25, wherein the crystalline form is characterized by an XRPD diffractogram having peaks expressed in degrees-2-theta at angles (±0.2 degrees) of 10.3, 17.3, 20.6, 21.1, 23.3, and 25.8.

28-29. (canceled)

30. A pharmaceutical composition comprising the crystalline form of claim 1 and a pharmaceutically acceptable carrier.

31. A method of inhibiting NLRP3 inflammasome in a subject in need thereof comprising administering to the subject a therapeutically effective amount of the crystalline form of claim 1.

32. A method of treating inflammation in a subject in need thereof comprising administering to the subject a therapeutically effective amount of the crystalline form of claim 1.

33. A method of treating inflammaging in a subject in need thereof comprising administering to the subject a therapeutically effective amount of the crystalline form of claim 1.

34. A method of treating cryopyrin-associated periodic syndrome (CAPS) in a subject in need thereof comprising administering to the subject a therapeutically effective amount of the crystalline form of claim 1.

35. A method of treating a disease or disorder of the inner ear in a subject in need thereof comprising administering to the subject a therapeutically effective amount of the crystalline form of claim 1.

36. The method of claim 35, wherein the disease or disorder of the inner ear is selected from the group consisting of hearing loss, hearing impairment, vertigo, Meniere's disease, and tinnitus.
